(19) United States
(12) Patent Application Publication
(10) Pub. No.: US 2010/0152301 A1
(43) Pub. Date: Jun. 17, 2010

(54) Treatment of Viral Infections

(75) Inventors: Yves Pommier, Bethesda, MD (US); Christophe Marchand, Gaithersburg, MD (US); Elena Semenova, Bethesda, MD (US); Allison Johnson, Rockville, MD (US)

Correspondence Address: EDWARDS ANGELL PALMER & DODGE LLP, P.O. BOX 55874, BOSTON, MA, 02205, US

(73) Assignee: GOVERNMENT OF THE US, AS REPRESENTED BY THE SECRETARY, DEPARTMENT OF HEALTH AND HUMAN SERVICES, ROCKVILLE, MD (US)

(21) Appl. No.: 12/085,895

(22) PCT Filed: Dec. 1, 2006
(86) PCT No.: PCT/US2006/046259

§ 371 Date: Jan. 8, 2010

Related U.S. Application Data
(60) Provisional application No. 60/741,769, filed on Dec. 1, 2005.

(51) Int. Cl. A61K 31/122; C12N 5/071; A61P 31/18

(52) U.S. Cl. 514/678; 435/325

(57) ABSTRACT

Treatment of cells or subjects (e.g., humans, animals) carrying or infected with a virus capable of causing an immunodeficiency disease by administration of one or more compounds that inhibit integrase.

### CROSS-REFERENCE TO RELATED APPLICATION

The present application claims priority from U.S. provisional application No. 60/741,769 filed Dec. 1, 2005, which is incorporated herein by reference in its entirety.

### STATEMENT REGARDING FEDERALLY SPONSORED RESEARCH OR DEVELOPMENT

Research supporting this application was carried out by the United States of America as represented by the Secretary, Department of Health and Human Services.

### BACKGROUND OF THE INVENTION

1. Field of the invention

The present invention relates to methods for treatment or prevention of an HIV infection and, more particularly, to use of one or more HIV-1 integrase inhibitor compounds to treat a subject suffering from or susceptible to an HIV infection.

2. Background

The human immunodeficiency virus type 1 (HIV-1, also referred to as HTLV-III LAV or HTLV-III/LAV) and, to a lesser extent, human immunodeficiency virus type 2 (HIV-2) is the etiological agent of the acquired immune deficiency syndrome (AIDS) and related disorders. Barre-Sinoussi, et al., Science, 220:868-871 (1983); Gallo, et al., Science, 224:500-503 (1984); Levy, et al., Science, 225:840-842 (1984); Popovic, et al., Science, 224:497-500 (1984); Sarngadharan, et al., Science, 224:506-508 (1984); Siegal, et al., N. Engl. J. Med., 305:1439-1444 (1981); Clavel, F., AIDS, 1-135-140. This disease is characterized by a long asymptomatic period followed by the progressive degeneration of the immune system and the central nervous system. Studies of the virus indicate that replication is highly regulated, and both latent and lytic infection of the CD4 positive helper subset of T-lymphocytes occur in tissue culture. Zagury, et al., Science, 231:850-853 (1986). The expression of the virus in infected patients also appears to be regulated as the titer of infectious virus remains low throughout the course of the disease. Both HIV-1 and 2 share a similar structural and function genomic organization, having regulatory genes such as tat, rev, net in addition to structural genes such as env, gag and pal.

While AIDS, itself, does not necessarily cause death, in many individuals the immune system is so severely depressed that various other diseases (secondary infections or unusual tumors) such as herpes, cytomegalovirus, Kaposi's sarcoma and Epstein-Barr virus related lymphomas among others occur, which ultimately results in death. These secondary infections may be treated using other medications. However, such treatment can be adversely affected by the weakened immune system. Some humans infected with the AIDS virus seem to live many years with little or no symptoms, but appear to have persistent infections. Another group of humans suffers mild immune system depression with various symptoms such as weight loss, malaise, fever and swollen lymph nodes. These syndromes have been called persistent generalized lymphadenopathy syndrome (PGL) and AIDS related complex (ARC) and may or may not develop into AIDS. In all cases, those infected with the HIV are believed to be persistently infective to others.

Integration is a crucial step in the virus life cycle of human immunodeficiency virus type 1 (HIV-1), and therefore inhibitors of HIV-1 integrase are candidates for antiretroviral therapy. Two 7-hydroxytropolone derivatives (α-hydroxytropolones) were found to inhibit HIV-1 integrase. A structure-activity relationship investigation with several tropolone derivatives demonstrated that the 7-hydroxy-group is useful for integrase inhibition. α-hydroxytropolones preferentially inhibit strand transfer and are inhibitory both in the presence of magnesium or manganese. Lack of inhibition of disintegration in the presence of magnesium coupled with results from different crosslinking assays is consistent with α-hydroxytropolones as interfacial inhibitors. It appears that α-hydroxytropolones chelate the divalent metal (Mg²⁺ or Mn²⁺) in the enzyme active site. One representative compound against HIV-1 integrase in biochemical assays (NSC 18806, IC₅₀=4.8±2.5 μM) exhibits cytoprotective activity against HIV-1_{IIIB} in a cell-based assay. α-Hydroxytropolones represent a new family of inhibitors for the development of novel drugs against HIV infection.

The screening and investigation of novel drugs against Human Immunodeficiency Virus (REV) remains critical because of the ongoing AIDS epidemics and of the fast emergence of virus variants resistant to present antiviral therapy (Kellerman et al., 2005). The replication steps of HIV, a member of the retrovirus family, are well known and can therefore be targeted rationally [for general review, see (De Clercq, 2005)]. After HIV binding to the host cell, viral single-stranded RNA genomes are released into the cell, and serve as templates for the virus-encoded reverse transcriptase to synthesize double-stranded DNA copies bearing the long terminal repeats (LTR) at both ends (Turner and Summers, 1999). The viral linear DNA is integrated into the host genome in a reaction catalyzed by the viral enzyme, integrase (IN). Integration is essential for viral replication as integrated viral DNA (provirus) serves as a template for the synthesis of new viruses after processing by the host cell transcription-translation machines (Asante-Appiah and Skalka, 1997; Brown, 1990; Fesen et al., 1993; Van Maele and Debyser, 2005).

Antiviral therapy currently involves the use of a combination of reverse transcriptase and HIV protease inhibitors of HIV. Recently inhibitors of virus fusion to the host cells have been developed (Barbaro et al., 2005; De Clercq, 2005). Since understanding that HIV integrase is crucial for virus replication, the search for integrase inhibitors has been ongoing (Debyser et al., 2002; Deprez et al., 2004; Fesen et al., 1993; Hazuda et al., 2000; Johnson et al., 2004; Pommier et al., 2005). Integrase inserts the proviral DNA into host chromosomes in two steps: 3′ processing (3′-P) and strand transfer (ST). 3′-P is an endonucleolytic cleavage reaction removing the 3′-ends of the viral LTR DNA (generally a dinucleotide pGpT for HIV-1) immediately 3′ from the conserved sequence (CA for HIV-1) (see FIG. 1A). ST is the insertion of the processed 3′-ends of the viral DNA into the cell genome (Asante-Appiah and Skalka, 1997). The HIV-1 integrase catalytic site contains three essential amino acids: Asp64, Asp116, and Glu152 (D,D-35 E-motif) that coordinate at least one and probably two divalent cations (Mg²⁺ or Mn²⁺) between the enzyme and its DNA substrates (Chiu and Davies, 2004; Engelman and Craigie, 1992).

The ST inhibitors 5-CITEP and L-731,988 have been proposed to chelate the divalent metal cations (Mg²⁺ or Mn²⁺) in the enzyme active site (Grobler et al., 2002; Marchand et al., 2003; Pommier et al., 2005). 5-CITEP has been co-crystallized in the catalytic domain of HIV integrase and shown to bind in the DDE motif (Goldgur et al., 1999). The diketo acid derivative L-731,988 was shown to block binding of target DNA in the integrase active site (Espeseth et al., 2000). The selective inhibition of the strand transfer reaction by diketo acids has been proposed to be due to their interfacial inhibition on preformed integrase-viral DNA complexes (Pommier and Marchand, 2005).

Tropolone derivatives are present in cupressaceous trees from genus Thuja and are probably responsible for resistance of fungal and insect attack on the heartwood (Baya et al., 2001; Diouf et al., 2002; Lim et al., 2005). Our experiments demonstrate the ability of the monomer 7-hydroxytropolones (α-hydroxytropolones) to preferentially inhibit the ST reaction by interfering with the enzyme catalytic site. α-Hydroxytropolone derivatives are new lead inhibitors for HIV-1 integrase.

It thus would be desirable to have a new compound that can treat cells infected with HIV. It would be particularly desirable to have a new therapy that can be used to treat cells by preventing integration of the virus.

### SUMMARY OF THE INVENTION

We have now discovered that certain compounds that inhibit HIV integrase, e.g., HIV-1 integrase inhibitor compounds, can be useful for treating cells infected by immunodeficiency viruses and methods of preventing cells from becoming infected by immunodeficiency viruses, preferably human immunodeficiency viruses such as HIV.

More particularly, we now provide therapeutic methods for treating or preventing disease or disease symptoms that in general comprise administration of a therapeutically effective amount of a compound that inhibits integrase (a HIV integrase inhibitor compound, or salt, solvate or hydrate thereof) to mammalian cells that are infected with an immunodeficiency virus, particularly a human immunodeficiency virus such as HIV.

The invention further methods that in general comprise administration of a therapeutically effective amount of a compound that inhibits integrase (e.g., an HIV integrase inhibitor) to a patient in need of treatment, such as a mammal suffering from or susceptible to an immunodeficiency virus, particularly a human immunodeficiency virus such as HIV.

A wide variety of integrase inhibitor compounds can be employed in the methods of the invention. For example, suitable compounds are reported herein, and various tropolone compounds are useful as such. In certain aspects, the integrase inhibitor compounds for use in the methods of the invention exhibit good activity in a standard in vitro integrase inhibition assay (including specifically the standard assays described herein).

Specifically preferred HIV integrase inhibitor compounds for use in accordance with the invention include tropolone compounds, α-hydroxy tropolone compounds, mono-hydroxy tropolone compounds, bis-hydroxy tropolone compounds, derivatives and prodrugs thereof, and salts, solvates, hydrates and polymorphs of all of the above.

Integrase inhibitor compounds used in accordance with the present invention (including methods delineated herein) can be any compound that inhibits integrase activity, including compounds of Formula (I), or salts, solvates, hydrates thereof:

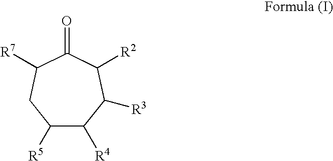

wherein,

each R¹ is independently F, Cl, Br, CF₃, NH₂, N(C₁-C₆ alkyl)₂, NO₂, CN, (C₁-C₆alkyl)O—, —OH, (C₁-C₆ alkyl)S(O)ₘ—, (C₁-C₆ alkyl)C(O)NH—, H₂N—C(NH)—, (C₁-C₆ alkyl)C(O)—, (C₁-C₆ alkyl)OC(O)—, N₃, (C₁-C₆ alkyl)OC(O)NR— and C₁-C₂₀ alkyl;

each R² is independently OH;

each R³ is independently H, alkyl, or R³ taken together with R⁴ and the carbon atoms to which they are each attached, respectively, form a cycloalkyl which may be optionally substituted with 1-4 R¹;

each R⁴ is independently H, alkyl, or R⁴ taken together with R³ and the carbon atoms to which they are each attached, respectively, form a cycloalkyl which may be optionally substituted with 1-4 R¹;

each R⁵ is independently H, alkyl or alkenyl;

each R⁷ is independently H or OH; and

each m is independently 0, 1 or 2.

Other aspects are those wherein the integrase inhibitor is one or more of the compounds of Formula (I) or salt, solvate or hydrate thereof, wherein R⁷ is OH; and wherein the integrase inhibitor is one or more of the compounds of Table 1 herein.

In certain embodiments, the compounds of the present invention can treat cells infected acutely and chronically by immunodeficiency viruses, for example, HIV, preferably HIV-1, and thus can be used to treat humans infected by HIV. For example, treatment of those diagnosed as having AIDS as well as those having ARC, PGL and those not yet exhibiting such conditions.

Another aspect is a method of reducing latent HIV-reservoirs in a subject including administration of an effective amount of one or more integrase inhibitor compounds.

Other aspects include a method of modulating strand transfer (ST) in an HIV-infected cell in a subject identified as in need of such treatment comprising administration to the subject of an effective amount of one or more integrase inhibitor compounds;

a method of inhibiting proviral DNA insertion into a host chromosome in an HIV-infected subject identified as in need of such treatment comprising administration to the subject of an effective amount of one or more integrase inhibitor compounds;

a method of inhibiting disulfide crosslinking in a subject identified as in need of such treatment comprising administration to the subject of an effective amount of one or more integrase inhibitor compounds;

a method of inhibiting HIV-1 integrase in a HIV-infected cell comprising administration to the cell of an effective amount of an HIV-1 integrase inhibitor such that the inhibition is mediated by chelation of Mg²⁺ or Mn²⁺ in the enzyme active site;

a method of inhibiting HIV-1 integrase in a subject comprising administration to the subject of an effective amount of an HIV-1 integrase inhibitor that is a more potent inhibitor of strand transfer than inhibitor of disintegration.

Other aspects of any of the methods herein are those wherein, the compound of Formula (I) is a α-hydroxytropolone compound that is capable of chelating the divalent metal (Mg²⁺ or Mn²⁺) in the enzyme active site;

wherein the integrase inhibitor compound is a α-hydroxytropolone compound that is capable of chelating the divalent metal (Mg²⁺ or Mn²⁺) in the enzyme active site;

wherein the enzyme active site comprises three essential amino acids: Asp64, Asp116, and Glu152 (D,D-35 E-motif) that coordinate at least one divalent cation (Mg²⁺ or Mn²⁺) between the enzyme and its DNA substrates; or

wherein the integrase inhibitor compound is a α-hydroxytropolone compound that is capable of interfacial inhibition on preformed integrase-viral DNA complexes.

The methods delineated herein include administering to a subject (e.g., a human or an animal) in need thereof an effective amount of one or more integrase inhibitors, e.g., compounds as delineated herein. The methods can also include the step of identifying that the subject is in need of treatment of diseases or disorders described herein, e.g., identifying that the subject is in need of integrase inhibition particularly in HIV-1 infected cells. The identification can be in the judgment of a subject or a health professional and can be subjective (e.g., opinion) or objective (e.g., measurable by a test or a diagnostic method). Tests for HIV infection are known in the art and include polymerase chain reaction-based (PCR-based) amplification and detection of viral RNA; Western blot detection of anti-HIV antibodies; agglutination assays for anti-HIV antibodies; ELISA-based detection of HIV-specific antigens (e.g., p24); line immunoassay (LIA); and other methods known to one of ordinary skill in the art. In each of these methods, a sample of biological material, such as blood, plasma, semen, or saliva, is obtained from the subject to be tested. Thus, the methods of the invention can include the step of obtaining a sample of biological material (such as a bodily fluid) from a subject; testing the sample to determine the presence or absence of detectable HIV infection, HIV particles, or HIV nucleic acids; and determining whether the subject is in need of treatment according to the invention, i.e., identifying whether the subject is in need of reactivation of a replication process or processes in latent HIV-infected cells.

The methods delineated herein can further include the step of assessing or identifying the effectiveness of the treatment or prevention regimen in the subject by assessing the presence, absence, increase, or decrease of a marker, including a marker or diagnostic measure of HIV infection, HIV replication, viral load, or expression of an HIV infection marker, preferably this assessment is made relative to a measurement made prior to beginning the therapy. Such assessment methodologies are known in the art and can be performed by commercial diagnostic or medical organizations, laboratories, clinics, hospitals and the like. As described above, the methods can further include the step of taking a sample from the subject and analyzing that sample. The sample can be a sampling of cells, genetic material, tissue, or fluid (e.g., blood, plasma, sputum, etc.) sample. The methods can further include the step of reporting the results of such analyzing to the subject or other health care professional. The method can further include additional steps wherein (such that) the subject is treated for the indicated disease or disease symptom.

In one aspect, the invention provides a method of treating HIV infection in a subject. The method comprises the steps of identifying a subject as in need of HIV integrase modulation (e.g., inhibition) in HIV-infected cells; and administrating of an effective amount of an inhibitor compound as described herein to the subject to modulate the viral replication process. In preferred embodiments, the HIV-1 integrase inhibitor is one or more of 18806, 310618, 43339, 89303, 18804, 18805, 43338, β-thujaplicinol, manicol, nootkatin, tropolone, β-thujaplicin, α-thujaplicin, γ-thujaplicin. In certain preferred embodiments, one or more peptidomimetic integrase inhibitor compounds are administered to the subject; in other preferred embodiments, one or more non-peptidomimetic integrase inhibitor compounds are administered to the subject. In certain preferred embodiments, the one or more integrase inhibitor compounds are of any one of the general formulae described herein. In certain embodiments, the administered integrase inhibitor compound has an IC50 of about 1000 nM or less in a standard in vitro HIV-1 integrase inhibition assay.

In another aspect, the invention provides a method of inhibiting HIV replication in a subject or a cell. The method comprises the steps of identifying a subject or cell as in need of modulation of replication processes in HIV-infected cells; administering an effective amount of an integrase inhibitor to the subject or cell to reactivate the viral replication process; and administering one or more HIV antiviral agents to the subject or cell to inhibit HIV viral replication.

In any of the above methods, in preferred embodiments, the method further comprises the step of administration of one or more additional therapeutic agents, e.g., anti-viral (e.g., anti-HIV) therapeutic agents, immunomodulators, or anti-infective agents, to the subject or cell. In preferred embodiments, the additional anti-viral agent(s) are a reverse transcriptase inhibitor (nucleoside or non-nucleoside), a protease inhibitor, another integrase inhibitor, or combination thereof.

In preferred embodiments of any of the methods described above, the cell is a lymphocytic cell or a monocytic cell. In preferred embodiments, the cell is a human cell, i.e., any human cell capable of sustaining a HIV virus.

The invention also provides pharmaceutical compositions comprising one or more integrase inhibitor compounds (including those described herein) and a suitable carrier therefore for use in the conditions referred to above.

The methods delineated herein can further include the step of assessing or identifying the effectiveness of the treatment or prevention regimen in the subject by assessing the presence, absence, increase, or decrease of a marker, including a marker or diagnostic measure of HIV infection, HIV replication, viral load, or expression of an HIV infection marker. Such assessment methodologies are known in the art and can be performed by commercial diagnostic or medical organizations, laboratories, clinics, hospitals and the like. The methods can further include the step of taking a sample from the subject and analyzing that sample. The sample can be a sampling of cells, genetic material, tissue, or fluid (e.g., blood, plasma, sputum, etc.) sample. The methods can further include the step of reporting the results of such analyzing to the subject or other health care professional.

Another aspect of the invention is a compound herein for use in the treatment or prevention in a subject of a disease, disorder or symptom thereof delineated herein.

Another aspect of the invention is the use of a compound herein in the manufacture of a medicament for treatment or prevention in a subject of a disease, disorder or symptom thereof delineated herein.

Other aspects of the invention are disclosed infra.

### BRIEF DESCRIPTION OF THE DRAWINGS

FIG. 1: Inhibition of HIV-1 integrase 3′-processing (3′-P) and strand transfer (ST) activities by NSC 18806. (A) Sequence of the 21 by oligonucleotide duplex that corresponds to the terminal U5 sequence of the HIV-1 LTR used as a substrate, and schematic representation of the integrase reactions. Arrowhead represents the 3′-P site. Asterisks represent the 5′-[³²P]-label. The initial step involves cleavage of two bases from the 3′-OH end resulting in a 19 bp product. ST products (STP) result from the covalent joining of the 3′-processed duplex into another identical duplex that serves as the target DNA. (B) PAGE analysis of HIV-1 integrase inhibition by NSC 18806 using the 21 bp duplex as substrate in the presence of Mg²⁺ or Mn²⁺. Drug concentrations are shown above each lane.

FIG. 2: Inhibition of HIV-1 integrase-catalyzed strand transfer (ST) by NSC 18806 is independent of 3′-processing. (A) Sequence of the preprocessed (19/21) oligonucleotide duplex used as substrate and schematic representation of the ST assay. Asterisks represent the 5′-[³²P]-label. Strand transfer products (STP) result from the covalent joining of the 3′-OH end of the precleaved substrate into another identical substrate that serves as the target DNA (B) PAGE analysis of HIV-1 integrase inhibition by NSC 18806 in the presence of Mg²⁺ or Mn²⁺. Drug concentrations are shown above each lane.

FIG. 3: Lack of inhibition of HIV-1 integrase-mediated disintegration by NSC 18806 in the presence of Mg²⁺. (A) Sequence of the oligonucleotides used as substrate for disintegration (Y-oligomer). The disintegration product results from cleavage of the 34-mer oligonucleotide and can be detected as a radiolabeled 19-bp oligonucleotide. Asterisks represent the 5′-[³²P]-label. (B) PAGE analysis of the HIV-1 integrase-mediated disintegration reactions in the presence of NSC 18806 and Mg²⁺ or Mn²⁺. Drug concentrations are shown above each lane.

FIG. 4: Summary and quantitative comparison of inhibition of the HIV-1 integrase reactions by NSC 18806 in the presence of Mg²⁺ (filled symbols) or Mn²⁺ (open symbols). (A) Reactions with the 21-bp duplex substrate (see FIG. 1A); 3′-processing (3′-P): triangles: strand transfer (ST): circles; (B) strand transfer assays with the preprocessed DNA substrate (see FIG. 2A); (C) Disintegration assays with the Y-substrate (see FIG. 3A). Data represent mean±SD for at least three independent experiments.

FIG. 5: The tropolone 7-hydroxy group can be important for inhibition of disulfide cross-linking between of HIV-1 integrase Q148 and the 5′-C of the DNA substrate. (A) Integrase-DNA crosslinking strategy. Left, schematic representation of the HIV-1 integrase cysteine (Cys) residues; Lower right, schematic representation of the mutant integrase used for crosslinking; Residue 148 on the flexible loop is mutated Q→C; cysteines 56, 65 and 280 are mutated to serine to eliminate non-specific crosslinking; Upper right, modified oligonucleotide used for crosslinking (DNA X-1) with a thioalkyl modification (Johnson et al., 2005). The crosslinked complex forms between the cysteine residue 148 and the 5′-C from the DNA X-1 substrate. (B) SDS-PAGE analysis of the crosslinking reaction showing metal-dependent inhibition by NSC 18804 and NSC 18806. The size of molecular weight markers in kDa is shown on the left. Drug concentrations in the reaction mixture are 1 mM. (C) Concentration-dependent inhibition of disulfide crosslinking by NSC 18806 in the presence of Mg²⁺ using the DNA X-1 substrate labeled with [³²P] at the 5′-end of the top strand.

FIG. 6: NSC 18806 does not interfere with HIV-1 integrase overall binding to viral DNA end in the Schiff base crosslinking assay. (A) Principle of the crosslinking assay. An abasic site is introduced by uracil DNA glycosylase in the DNA substrate containing uracil at the position corresponding to the adenine in the conserved CA-dinucleotide. The asterisks indicate the 5′-[³²P]-label. An integrase nitrogen nucleophile (probably lysine) attacks the C1′-carbon of the abasic site (Mazumder and Pommier, 1995). Rearrangement of the initial enzyme-DNA complex leads to the formation of a Schiff base intermediate that can be stabilized by NaBH₄. (B) SDS-PAGE analysis showing no inhibition by the various tropolone derivatives on the crosslinking reactions between integrase and DNA in the presence of Mg²⁺.

FIG. 7: Activity of NSC 18806 against the cytopathic action of HIV-1_{IIIB} on lymphoid MT-2 cells. Effects of NSC 18806 on HIV-infected (filled circle) and mock-infected cells (open square).

FIG. 8: Proposed model of action α-hydroxytropolones against HIV-1 integrase. NSC 18806 is shown bound at the integrase-DNA complex chelating the divalent metals (Mg²⁺ or Mn²⁺) in the integrase active site. Me²⁺: divalent cation.

### DETAILED DESCRIPTION OF THE INVENTION

It has been discovered that integrase inhibitors, e.g., compounds of the formulae herein, can be used to modulate viral replication processes in cells infected by an immunodeficiency virus, preferably human cells infected with HIV and thus can be used for treatment in HIV-infected individuals.

The methods of the invention in general comprise administration of a therapeutically effective amount of a compound that inhibits integrase (a integrase inhibitor; e.g., an HIV integrase inhibitor, an HIV-1 integrase inhibitor, a compound of as delineated herein) to a patient in need of treatment, such as a mammal suffering from or susceptible to an immunodeficiency virus, particularly a human immunodeficiency virus such as HIV (e.g., HIV-1).

The compounds and pharmaceutical compositions of the present invention are useful for inhibiting HIV integrase, preventing infection by HIV, treating infection by HIV, delaying the onset of AIDS, and treating AIDS, in adults, children or infants. Delaying the onset of AIDS, treating AIDS, or preventing or treating infection by HIV is defined as including, but not limited to, treating a wide range of states of HIV infection: AIDS, AIDS-related complex (ARC), both symptomatic and asymptomatic, and actual or potential exposure to HIV. For example, the compounds and pharmaceutical compositions thereof of this invention are useful in treating infection by HIV after suspected past exposure to HIV by, e.g., blood transfusion, exchange of body fluids, bites, accidental needle stick, or exposure to patient blood during surgery.

Particularly preferred integrase inhibitor compounds for use in the methods of the invention exhibit good activity in a standard in vitro integrase inhibition assay, preferably an IC₅₀ (concentration required to inhibit integrase activity by 50% relative to control) in such an assay of about 1000 nM or less, (e.g., an IC₅₀ about 100 nM or less, an IC₅₀ about 50 nM or less, an IC₅₀ about 25 nM or less, an IC₅₀ about 10 nM or less. References herein to a standard in vitro integrase inhibition assay are also described herein and in references delineated herein.

Specifically integrase inhibitor compounds for use in the methods of the invention include the following delineated compounds (including those of Table 1) where the compound is structurally depicted or is listed with one or more chemical names thereof (i.e. one or both of an IUPAC-type name and other designator are listed); and pharmaceutically acceptable, solvates, derivatives or prodrugs of the compounds. Such compounds include, for example, β-thujaplicinol, manicol, nootkatin, tropolone, β-thujaplicin, α-thujaplicin, and γ-thujaplicin.

TABLE 1
Inhibition by tropolones of the different activities of HIV-1 integrase.

| | In vitro IC₅₀* Values (μM) | | | | |
|---|---|---|---|---|---|
| Structure; | 21 bp substrate | | | Preprocessed | |
| (NCS #) | | 3′-P | ST | substrate | Disintegration |
| 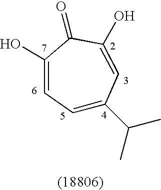 | Mg²⁺ Mn²⁺ | 117.3 ± 7.1 24.6 ± 3.9 | 21.6 ± 3.4 4.8 ± 2.5 | 18.1 ± 6.2 5.0 ± 2.9 | >333² 23.8 ± 5.9 |
| 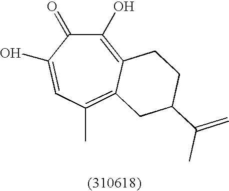 | Mg²⁺ Mn²⁺ | 182.0 ± 42.5 20.2 ± 8.9 | 71.1 ± 3.8 11.7 ± 5.2 | >333³ 25.8 ± 6.6 | >333² 98.3 ± 30.9 |
| 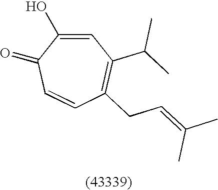 | Mg²⁺ Mn²⁺ | >333² >333² | >333³ >333³ | >333² >333³ | >333¹ >333¹ |
| 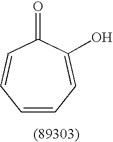 | Mg²⁺ Mn²⁺ | >333¹ >333² | >333³ >333³ | >333² >333² | >333¹ >333¹ |
| 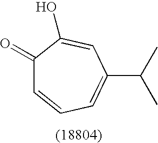 | Mg²⁺ Mn²⁺ | >333³ >333³ | >333² >333² | >333² >333² | >333¹ >333¹ |
| 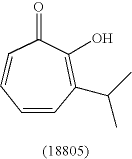 | Mg²⁺ Mn²⁺ | >333² >333² | >333¹ >333¹ | >333¹ >333¹ | >333¹ >333¹ |
| 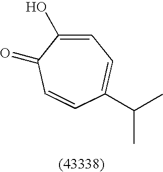 | Mg²⁺ Mn²⁺ | >333¹ >333¹ | >333¹ >333¹ | >333¹ >333¹ | >333¹ >333¹ |

All data represent mean values and standard deviations for at least three independent experiments.
*Concentration required for 50% inhibition of HIV-1 integrase activity in the different assays
¹inhibition activity ≦ 5%;
²5% < inhibition activity < 20%;
³20% < inhibition activity < 40% at drug concentration of 333 μM.

The efficacy of any particular integrase inhibitor in the therapeutic methods of the invention can be readily determined. For example, compounds with superior intrinsic inhibitory activity against and selectivity for HIV-integrase can be identified through the in vitro assays discussed above and herein.

The inhibitor compounds suitable for use in the methods of the invention may have asymmetric centers and occur as racemates, racemic mixtures, and as individual diastereomers, with all possible isomers, including optical isomers, being included in the present invention. Unless otherwise specified, named amino acids are understood to have the natural “L” stereoconfiguration. Further, inhibitor compounds suitable for use in the methods of the invention may have enol form and keto form tautomers, depending upon the form of its substituents. The compounds of the present invention includes such enol form and keto form isomers and their mixtures.

Another aspect is a isotopologue compound of any of the formulae delineated herein. Such compounds have one or more isotopic atoms or heavy atom isotopes (e.g., ³H, ²H, ¹⁴C, ¹³C, ³⁵S, ¹²⁵I, ¹³¹I) introduced into (or in place of a natural abundance atom) the compound. Such compounds are useful for drug metabolism studies and diagnostics.

The following definitions apply to the above-discussed compounds, including those of the above general formulae herein:

“Alkyl” is intended to include both branched and straight-chain saturated aliphatic hydrocarbon groups having the specified number of carbon atoms. “Cycloalkyl” is intended to include non-aromatic cyclic hydrocarbon groups having the specified number of carbon atoms. Examples of cycloalkyl groups include cyclopropyl, cyclobutyl, cyclopentyl, cyclohexyl and the like. “Alkenyl” groups include those groups having the specified number of carbon atoms and having one or several double bonds. Examples of alkenyl groups include vinyl, allyl, isopropenyl, pentenyl, hexenyl, heptenyl, cyclopropenyl, cyclobutenyl, cyclopentenyl, cyclohexenyl, 1-propenyl, 2-butenyl, 2-methyl-2-butenyl, isoprenyl, farnesyl, geranyl, and the like. As used herein, “aryl” is intended to include any stable monocyclic, bicyclic or tricyclic carbon ring(s) of up to 7 members in each ring, wherein at least one ring is aromatic. Examples of aryl groups include phenyl, naphthyl, anthracenyl, biphenyl, tetrahydronaphthyl, indanyl, phenanthrenyl and the like. The term heterocycle or heterocyclic, as used herein, represents a stable 5 to 7 membered monocyclic or stable 8 to 11 membered bicyclic or stable 11-membered tricyclic heterocycle ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothio-pyranyl sulfone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, 2-oxopiperazinyl, 2-oxopiperidinyl, 2-oxopyrrolidinyl, piperidyl, piperazinyl, pyridyl, pyridyl N-oxide, pyridonyl, pyrazinyl, pyrazolidinyl, pyrazolyl, pyrimidinyl, pyrrolidinyl, pyrrolyl, quinazolinyl, quinolinyl, quinolinyl N-oxide, quinoxalinyl, tetrahydrofuryl, tetrahydroisoquinolinyl, tetrahydro-quinolinyl, thiamorpholinyl, thiamorpholinyl sulfoxide, thiazolyl, thiazolinyl, thienofuryl, thienothienyl, and thienyl.

As used herein, the term “substituted” in reference to any chemical functional group is intended to include that group which is substituted with 1 or more (e.g., 2, 3, 4, 5, 6, 7, 8, 9, 10, etc.) substituents selected from the group which includes but is not limited to F, Cl, Br, CF₃, NH₂, N(C₁-C₆ alkyl)₂, NO₂, CN, (C₁-C₆ alkyl)O—, —OH, (C₁-C₆ alkyl)S(O)ₘ—, (C₁-C₆ alkyl)C(O)NH—, H₂N—C(NH)—, (C₁-C₆ alkyl)C(O)—, (C₁-C₆ alkyl)OC(O)—, N₃, (C₁-C₆ alkyl)OC(O)NR— and C₁-C₂₀ alkyl.

The pharmaceutically acceptable salts of inhibitor compounds for use in the methods of the invention include known non-toxic salts, e.g. pharmaceutically acceptable inorganic or organic acids such as the following acids: hydrochloric, hydrobromic, sulfuric, sulfamic, phosphoric, nitric, acetic, propionic, succinic, glycolic, stearic, lactic, malic, tartaric, citric, ascorbic, pamoic, maleic, hydroxymaleic, phenyl-acetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. The pharmaceutically acceptable salts of inhibitor compounds for use in the methods of the invention can be synthesized from the corresponding inhibitor of this invention which contain a basic moiety by conventional chemical methods. Generally, the salts are prepared by reacting the free base with stoichiometric amounts or with an excess of the desired salt-forming inorganic or organic acid in a suitable solvent or various combinations of solvents.

The heteroaromatic ring group means a 5-membered or 6-membered monocyclic aromatic heterocyclic group containing one or two heteroatoms, which are the same or different, selected from the group consisting of an oxygen atom, a nitrogen atom and a sulfur atom, or a fused aromatic heterocyclic group having such a monocyclic aromatic heterocyclic group fused with the above-mentioned aryl group or having the same or different such monocyclic aromatic heterocyclic groups fused with each other, which may, for example, be a pyrrolyl group, an imidazolyl group, a pyrazolyl group, a pyridyl group, a pyrazinyl group, a pyrimidinyl group, a pyridazinyl group, an oxazolyl group, an isoxazolyl group, a furyl group, a thienyl group, a thiazolyl group, an isothiazolyl group, an indolyl group, a benzofuranyl group, a benzothienyl group, a benzimidazolyl group, a benzoxazolyl group, a benzisoxazolyl group, a benzothiazolyl group, a benzisothiazolyl group, an indazolyl group, a purinyl group, a quinolyl group, an isoquinolyl group, a phthalazinyl group, a naphthylidinyl group, a quinoxalinyl group, a quinazolinyl group, a cinnolinyl group or a pteridinyl group. Among them, a furyl group, a thienyl group, a pyridyl group, a pyrimidinyl group, an oxazolyl group, an isoxazolyl group, a thiazolyl group, a benzofuranyl group, a benzothienyl group, a benzimidazolyl group, a benzoxazolyl group, a benzothiazolyl group or a quinolyl group is preferred. The lower alkyl group means a C₁₋₆ linear or branched alkyl group, which may, for example, be a methyl group, an ethyl group, a propyl group, an isopropyl group, a butyl group, a sec-butyl group, a tert-butyl group, a pentyl group or a hexyl group. Among them, a methyl group or an ethyl group is preferred. The lower hydroxyalkyl group means the above-mentioned lower alkyl group having a hydroxyl group, i.e. a C₁₋₆ hydroxyalkyl group, such as a hydroxymethyl group, a hydroxyethyl group, a hydroxypropyl group or a hydroxybutyl group. Among them, a hydroxymethyl group or a hydroxyethyl group is preferred. The lower alkoxy group means a C₁₋₆ alkoxy or alkylenedioxy group, which may, for example, be a methoxy group, an ethoxy group, a propoxy group, an isopropoxy group, a butoxy group, a tert-butoxy group, a methylenedioxy group, an ethylenedioxy group or a trimethylenedioxy group. Among them, a methoxy group, an ethoxy group or a methylenedioxy group is preferred. The lower carboxyalkyl group means the above-mentioned lower alkyl group having a carboxyl group, i.e. a C₁₋₇ carboxyalkyl group, such as a carboxymethyl group, a carboxyethyl group, a carboxypropyl group or a carboxybutyl group. Among them, a carboxymethyl group or a carboxyethyl group is preferred. The aralkyl group means the above-mentioned lower alkyl group having the above-mentioned aryl group, such as a benzyl group, a phenethyl group, a 3-phenylpropyl group, a 1-naphthylmethyl group, a 2-naphthylmethyl group or a 1-(2-naphthyl)ethyl group. Among them, a benzyl group, a phenethyl group or a 2-naphthylmethyl group is preferred. The saturated aliphatic hydrocarbon group may, for example, be an ethylene group, a trimethylene group, a tetramethylene group, a pentamethylene group, a hexamethylene group, a heptamethylene group or an octamethylene group. For example, a trimethylene group, a tetramethylene group or a pentamethylene group is preferred.

The unsaturated aliphatic hydrocarbon group means an unsaturated aliphatic hydrocarbon group having one or more, preferably one or two double bonds, at optional position(s) on the carbon chain, which may, for example, be a vinylene group, a propenylene group, a 1-butenylene group, a 2-butenylene group, a 1,3-butadienylene group, a 1-pentenylene group, a 2-pentenylene group, a 1,3-pentadienylene group, a 1,4-pentadienylene group, a 1-hexenylene group, a 2-hexenylene group, a 3-hexenylene group, a 1,3-hexadienylene group, a 1,4-hexadienylene group, a 1,5-hexadienylene group, a 1,3,5-hexatrienylene group, a 1-heptenylene group, a 2-heptenylene group, a 3-heptenylene group, a 1,3-heptadienylene group, a 1,4-heptadienylene group, a 1,5-heptadienylene group, a 1,6-heptadienylene group, a 1,3,5-heptatrienylene group, a 1-octenylene group, a 2-octenylene group, a 3-octenylene group, a 4-octenylene group, a 1,3-octadienylene group, a 1,4-octadienylene group, a 1,5-octadienylene group, a 1,6-octadienylene group, a 1,7-octadienylene group, a 2,4-octadienylene group, a 2,5-octadienylene group, a 2,6-octadienylene group, a 3,5-octadienylene group, a 1,3,5-octatrienylene group, a 2,4,6-octatrienylene group or a 1,3,5,7-octatetraenylene group. Among them, a propenylene group, a 1-butenylene group, a 1,3-butadienylene group or a 1-pentenylene group is preferred. The halogen atom may be a fluorine atom, a chlorine atom, a bromine atom or an iodine atom. For example, a fluorine atom or a chlorine atom is preferred.

The lower alkoxycarbonyl group means a C₁₋₇ alkoxycarbonyl group, such as a methoxycarbonyl group, an ethoxycarbonyl group, a propoxycarbonyl group, a butoxycarbonyl group or a tert-butoxycarbonyl group. Among them, a methoxycarbonyl group or an ethoxycarbonyl group is preferred. The lower alkylcarbamoyl group means a carbamoyl group mono-substituted or di-substituted by the above-mentioned lower alkyl group, such as a methylcarbamoyl group, an ethylcarbamoyl group, a dimethylcarbamoyl group or a diethylcarbamoyl group. The lower fluoroalkyl group means the above-mentioned lower alkyl group having fluorine atom(s), i.e. a C₁₋₆ fluoroalkyl group, such as a fluoromethyl group, a difluoromethyl group, a trifluoromethyl group, a 1-fluoroethyl group, a 2-fluoroethyl group, a 2,2,2-trifluoroethyl group or a pentafluoroethyl group.

The base-addition salt may, for example, be an alkali metal salt such as a sodium salt or a potassium salt; an alkaline earth metal salt such as a calcium salt or a magnesium salt; an ammonium salt; or an organic amine salt such as a trimethylamine salt, a triethylamine salt, a dicyclohexylamine salt, an ethanolamine salt, a diethanolamine salt, a triethanolamine salt, a procaine salt or an N,N′-dibenzylethylenediamine salt. The acid-addition salt may, for example, be an inorganic acid salt such as a hydrochloride, a sulfate, a nitrate, a phosphate or a perchlorate; an organic acid salt such as a maleate, a fumarate, a tartrate, a citrate, an ascorbate or a trifluoroacetate; or a sulfonic acid salt such as a methanesulfonate, an isethionate, a benzenesulfonate or a p-toluenesulfonate.

While compounds having the precise structure of the formulae herein are preferred, as the terms are defined herein compounds coming within the formulae herein include structurally related compounds, including those compounds that are pharmaceutically acceptable salts, solvates, hydrates, and prodrugs of compounds delineated herein. Examples of prodrugs include esters and other pharmaceutically acceptable derivatives, which, upon administration to a subject, are capable of providing the parent compounds described herein (see Goodman and Gilman's, The Pharmacological basis of Therapeutics, 8th ed., McGraw-Hill, Int. Ed. 1992, “Biotransformation of Drugs”). As used herein and unless otherwise indicated, the term “prodrug” means a derivative of a compound that can hydrolyze, oxidize, or otherwise react under biological conditions (in vitro or in vivo) to provide a compound of this invention. Prodrugs may only become active upon such reaction under biological conditions, or they may have activity in their unreacted forms.

Some integrase inhibitor compounds may have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms. All such isomeric forms of these compounds are expressly included in the present invention. The compounds of this invention may also be represented in multiple tautomeric forms, in such instances, the invention expressly includes all tautomeric forms of the compounds described herein. All such isomeric forms of such compounds are expressly included in the present invention. All crystal forms of the compounds described herein are expressly included in the present invention.

Hence, in one preferred embodiment the present invention can be used in treating those diagnosed as having AIDS as well as those having ARC, PGL and those seropositive but asymptomatic patients. For example, as a preventative, an effective amount of an integrase inhibitor compound can also be used prophylactically as a preventative for high risk individuals.

Compounds of the present invention can be used to treat cells, especially mammalian cells and in particular human cells, infected by an immunodeficiency virus such as HIV. As a result of treatment with compounds of the present invention the number of latently infected cells can be significantly reduced.

The compounds of the present invention can be administered to HIV infected individuals or to individuals at high risk for HIV infection, for example, those having sexual relations with an HIV infected partner, intravenous drug users, etc. Because of their effect of inducing lytic viral replication, the compounds of the present invention and pharmaceutical compositions comprising one or more compounds of formula I can be used prophylactically as a method of prevention for such individuals to minimize their risk of cells becoming latently infected. The compound is administered in an effective amount as set forth below by methodology such as described herein.

In general for the treatment of immunodeficiency viral infections, for example an HIV infection, a preferred effective dose of one or more therapeutic compounds can be readily determined based on known factors such as efficacy of the particular therepautic agent used, age, weight and gender of the patient, and the like. See dosage guidelines as set forth e.g. in Remington, The Science and Practice of Pharmacy, 20^{th} Edition. For certain preferred dosages, an integrase inhibitor compound may be administered to a mammal (e.g. human) in the range 0.1 mg to 5 g per kilogram body weight of recipient per day, more preferably in the range of 0.1 mg to 1,000 mg per kilogram body weight per day, and still more preferably in the range of 1 to 600 mg per kilogram of body weight per day. The desired dose is suitably administered once or several more sub-doses administered at appropriate intervals throughout the day, or other appropriate schedule.

Preferably a therapeutic compound (e.g., a compound of the formulae herein) used in accordance with the invention will be in an isolated form distinct as it may be naturally found and in a comparatively pure form, e.g., at least 85% by weight pure, more preferably at least 95% pure. For some treatments in accordance with the present invention, it may be desirable that administered compound of Formula I be at least 98% or even greater than 99% pure. Such a material would be considered sterile for pharmaceutical purposes. Potential contaminants include side products that may result upon synthesis of a compound of the invention or materials that may be otherwise associated with the compound prior to its isolation and purification. The present compounds should preferably be sterile and pyrogen free. Purification techniques known in the art may be employed, for example chromatography.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey, ape, monkey, or human), and more preferably a human. In one embodiment, the subject is an immunocompromised or immunosuppressed mammal, preferably a human (e.g., an HIV infected patient). In another embodiment, the subject is a farm animal (e.g., a horse, a cow, a pig, etc.) or a pet (e.g., a dog or a cat). In a preferred embodiment, the subject is a human.

Administration of the compounds of the invention may be by any suitable route including oral, rectal, nasal, topical (including buccal and sublingual), vaginal and parenteral (including subcutaneous, intramuscular, intravenous and intradermal) with oral or parenteral being preferred. It will be appreciated that the preferred route may vary with, for example, the condition and age of the recipient.

The administered ingredients may be used in therapy in conjunction with other medicaments such as reverse transcriptase inhibitors such as dideoxynucleosides, e.g. zidovudine (AZT), 2′,3′-dideoxyinosine (ddI) and 2′,3′-dideoxycytidine (ddC), lamivudine (3TC), stavudine (d4T), and TRIZIVIR (abacavir+zidovudine+lamivudine), nonnucleosides, e.g., efavirenz (DMP-266, DuPont Pharmaceuticals/Bristol Myers Squibb), nevirapine (Boehringer Ingleheim), and delaviridine (Pharmacia-Upjohn), TAT antagonists such as Ro 3-3335 and Ro 24-7429, protease inhibitors, e.g., AGENERASE (GlaxoSmithKline), indinavir (Merck), ritonavir (Abbott), saquinavir (Hoffmann LaRoche), nelfinavir (Agouron Pharmaceuticals), 141 W94 (Glaxo-Wellcome), atazanavir (Bristol Myers Squibb), amprenavir (GlaxoSmithKline), fosamprenavir (GlaxoSmithKline), tipranavir (Boehringer Ingleheim), KALETRA (lopinavir+ritonavir, Abbott) and other agents such as 9-(2-hydroxyethoxymethyl)guanine (acyclovir), interferon, e.g., alpha-interferon, interleulcin II, and phosphonoformate (Foscamet) or in conjunction with other immune modulation agents or treatments including bone marrow or lymphocyte transplants or other medications such as levamisol or thymosin which would increase lymphocyte numbers and/or function as is appropriate. Additionally, an integrase inhibitor compound may be administered in coordination or conunction with an entry inhibitor e.g. T20 (enfuvirtide, Roche/Trimeris) or UK-427,857 (Pfizer). Because many of these drugs are directed to different targets, e.g., viral integration, it is anticipated that an additive or synergistic result will be obtained by this combination.

In one embodiment, one or more compounds of the formulae herein are used in conjunction with one or more therapeutic agents useful for treatment or prevention of HIV, a symptom associated with HIV infection, or other disease or disease symptom such as a secondary infection or unusual tumor such as herpes, cytomegalovirus, Kaposi's sarcoma and Epstein-Barr virus-related lymphomas among others, that can result in HIV immuno-compromised subjects.

In certain embodiments, the treatment methods herein include administration of a so-called HIV-drug “cocktail” or combination therapy, wherein a combination of reverse transcriptase inhibitor(s) and HIV protease inhibitor(s) is co-administered. In a preferred embodiment, a highly active anti-retroviral therapy (HAART) treatment regime is combined with treatment with an integrase inhibitor according to the invention.

In certain embodiments, the methods involve modulation of any gene that exhibits altered expression in chronically HIV-infected cells compared to uninfected parental cell. The methods herein can involve, or target, any of the genes listed herein. This modulation can be direct or indirect, that is, it can be by direct control of expression or binding activity of the target, or by indirect control of the expression or binding activity of the target.

The present invention includes use of both racemic mixtures and optically active stereoisomers of the integrase inhibitor compounds.

One or more integrase inhibitor compounds may be administered alone, or as part of a pharmaceutical composition, comprising at least one integrase inhibitor compound together with one or more acceptable carriers thereof and optionally other therapeutic ingredients, including those therapeutic agents discussed above. The carrier(s) should be “acceptable” in the sense of being compatible with the other ingredients of the formulation and not deleterious to the recipient thereof.

Compositions of the compounds of the invention (e.g., compounds of the formulae herein) used in combination with other compounds (e.g., reverse transcriptase inhibitors, protease inhibitors, integrase inhibitors and the like) may be employed alone or in combination with acceptable carriers such as those described below. For the treatment of immunodeficiency viral infections, for example an HIV infection, a suitable effective dose of a compound in such a composition will be in the range of 1 to 5,000 mg per kilogram body weight of recipient per day, preferably in the range of 10 to 4,000 mg per kilogram body weight of recipient per day. When multiple compounds having complementary activity are administered together it is expected one can use the lower portion of these ranges (or even less).

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., formulation into therapeutic products, intermediates for use in production of therapeutic compounds, isolatable or storable intermediate compounds). The compounds delineated herein are commercially available or readily synthesized by one of ordinary skill in the art using methodology known in the art.

The compositions include those suitable for oral, rectal, nasal, topical (including buccal and sublingual), vaginal or parenteral (including subcutaneous, intramuscular, intravenous and intradermal) administration. The formulations may conveniently be presented in unit dosage form, e.g., tablets and sustained release capsules, and in liposomes, and may be prepared by any methods well known in the art of pharmacy.

Such methods include the step of bringing into association the to be administered ingredients with the carrier which constitutes one or more accessory ingredients. In general, the compositions are prepared by uniformly and intimately bringing into association the active ingredients with liquid carriers, liposomes or finely divided solid carriers or both; and then if necessary shaping the product.

Compositions of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

Compositions suitable for topical administration include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Compositions suitable for topical administration to the skin may be presented as ointments, creams, gels and pastes comprising one or more compounds of the present invention and a pharmaceutically acceptable carrier. A suitable topical delivery system is a transdermal patch containing the ingredient to be administered.

Compositions suitable for rectal administration may be presented as a suppository with a suitable base comprising, for example, cocoa butter or a salicylate.

Compositions suitable for nasal administration wherein the carrier is a solid include a coarse powder having a particle size, for example, in the range 20 to 500 microns which is administered in the manner in which snuff is taken, i.e., by rapid inhalation through the nasal passage from a container of the powder held close up to the nose. Suitable formulations wherein the carrier is a liquid, for administration, as for example, a nasal spray or as nasal drops, include aqueous or oily solutions of the active ingredient.

Compositions suitable for vaginal administration may be presented as pessaries, tampons, creams, gels, pastes, foams or spray formulations containing in addition to the active ingredient such carriers as are known in the art to be appropriate.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets of the kind previously described.

It should be understood that in addition to the ingredients particularly mentioned above the formulations of this invention may include other agents conventional in the art having regard to the type of formulation in question, for example, those suitable for oral administration may include flavoring agents.

The use of the term “or” is unless otherwise indicated, to be construed as being inclusive. That is, the recitation of A, B or C is meant include A, B or C each alone, or in any combination (e.g., A and B, B and C, A and C, and A and B and C) thereof.

All documents mentioned herein (including patents, patent applications, and other references) are incorporated herein by reference.

The present invention is further illustrated by the following examples. These examples are provided to aid in the understanding of the invention and are not to be construed as limitations thereof.

### MATERIALS AND METHODS

### Example 1

Compounds. All drugs were obtained from the National Cancer Institute chemical repository from the Developmental Therapeutics Program (DTP, NCI, NIH, Bethesda, Md.). Compounds were dissolved in 100% DMSO. Stock solutions (10 mM) were stored at −20° C.

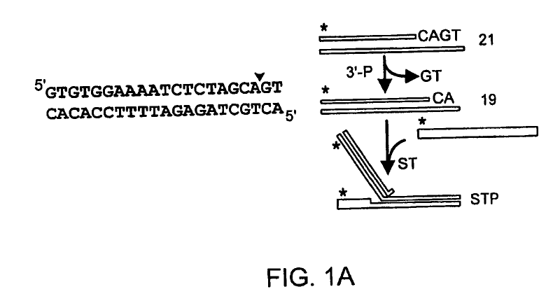

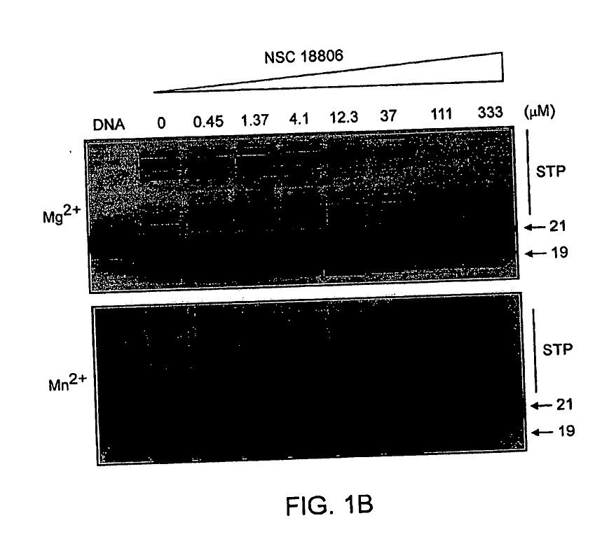

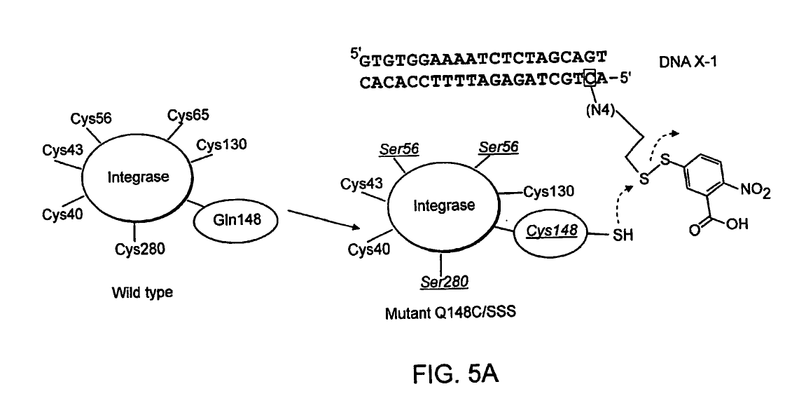

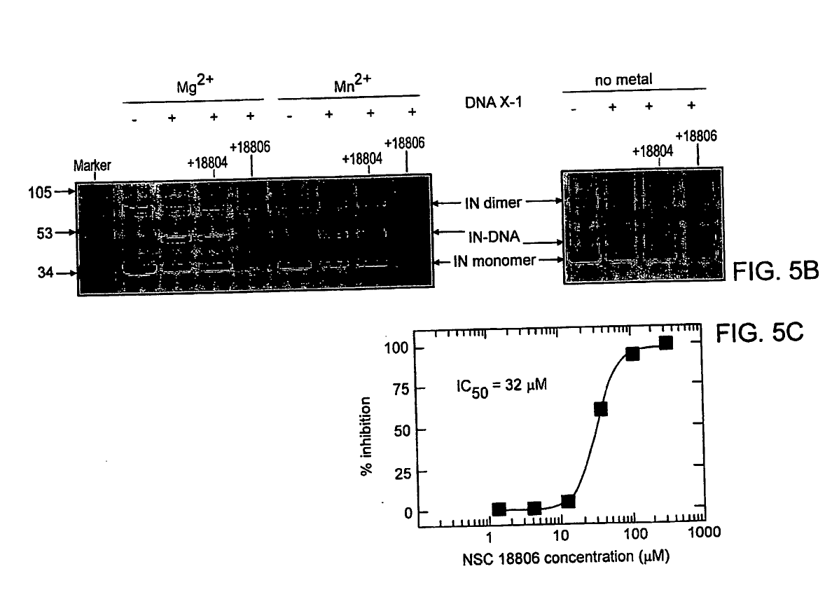

Recombinant HIV integrase and oligonucleotide substrates. Expression and purification of the recombinant HIV-1 integrase in Escherichia coli were performed according to (Leh et al., 2000; Marchand et al., 2001) with addition of 10% glycerol to all buffers. The preparation of the Q148C/SSS-mutant integrase will be described elsewhere (Johnson et al., 2005). The oligonucleotide substrates, except those used for the disulfide crosslinking (FIG. 5A), were purchased from Integrated DNA Technologies, Inc. (Coraville, Iowa) and purified by polyacrylamide gel. The sequences of DNA substrates are shown in FIGS. 1A, 2A, 3A and 6A. The single-stranded oligonucleotides were 5′-end labeled with [γ³²P]-ATP (Perkin Elmer, Wellesley, M A) and T₄ polynucleotide kinase (New England BioLabs, Ipswich, M A). Unincorporated nucleotide was removed using mini Quick Spin Oligo columns (Roche Diagnostics, Indianapolis, Ind.). Substrates were obtained after annealing with complementary non-labeled oligonucleotides. The thiol-modified substrate (FIG. 5A) for disulfide crosslinking assay was synthesized as essentially described by W. Santos and G. Verdine (Department of Chemistry and Chemical Biology, Harvard University, Cambridge, Mass.) (He and Verdine, 2002).

Integrase catalytic assays. Reactions were performed in 10 μl with 300 nM of recombinant IN, 20 nM of the 5′-end [³²P]-labeled oligonucleotide substrates and inhibitors at the indicated concentrations. 10% DMSO was included in controls. Reactions were incubated for 40 min at 37° C. in a buffer containing a final concentration of 25 mM MOPS, pH 7.2, 25 mM NaCl, 14.3 mM β-mercaptoethanol and 7.5 mM of divalent cations (MgCl₂ or MnCl₂ as indicated). Reactions were stopped by addition of 20 μl loading dye (10 mM EDTA, 98% deionized formamide, 0.025% xylene cyanol and 0.025% bromophenol blue). Reactions were heated at 95° C. for 1 min before electrophoresis in 20% polyacrylamide-7 M urea gels. Gels were dried and reaction products were visualized and quantitated with a Molecular Dynamics PhosphorImager (Sunnyvale, Calif.). Densitometric analyses were performed using ImageQuant from the Molecular Dynamics software. The concentrations at which enzyme activity was reduced by 50% (IC₅₀), was determined using “Prism” software (GraphPad Software, San Diego, Calif.) for nonlinear regression to fit dose-response data to logistic curve models.

Integrase binding to HIV DNA using the disulfide-crosslinking assay The disulfide crosslinking assay is essentially as described in Johnson et al. (Johnson et al., 2005). Briefly, 10 μM recombinant Q148C/SSS-mutant integrase was incubated with 10 μM DNA substrate (FIG. 5A) containing tethered thiols in the presence of 20 mM Tris, pH 7.4, 10% glycerol and 7.5 mM of divalent cations (MgCl₂ or MnCl₂ as indicated) for 20 min at 37° C. Reactions were stopped by the addition of 20 mM methylmethanethiosulfonate (capping reagent). Non-reducing gel loading buffer (100 mM Tris-HCl, pH 6.8, 4% SDS, 0.2% bromophenol blue, 20% glycerol) was added and samples were heated at 95° C. before loading onto 16% tricine gels (Invitrogen, Carlsbad, Calif.). Gels were stained with Microwave Blue according to manufacturer's recommendations (Protiga, Frederick, Md.).

Alternatively, for dose response experiments, 500 nM integrase was incubated with NSC 18806 as shown at FIG. 5C in the buffer described above for 20 minutes. DNA (20 nM) containing a [5′-³²P]-label on one strand and a thiol-modified cytosine on the other strand was added and reactions were capped with methylmethanethiosulfonate at 1 minute. Following capping, non-reducing gel loading buffer (100 mM Tris-HCl, pH 6.8, 4% SDS, 0.2% bromophenol blue, 20% glycerol) was added and samples were directly loaded on 16% tricine gels (Invitrogen, Carlsbad, Calif.). Gels were dried and reaction products were quantitated using the same way as described above.

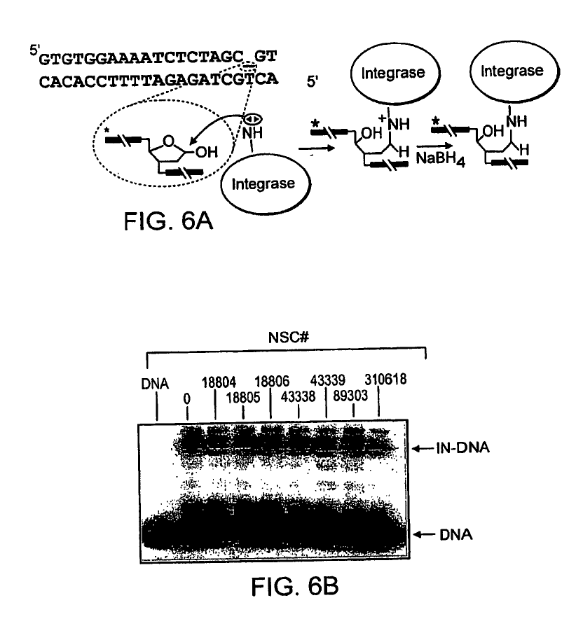

Integrase binding to HIV DNA using the shiff-base assay. The shiff-base assay was performed as described (Mazumder and Pommier, 1995). Briefly, 300 nM recombinant IN was incubated with inhibitors (at the indicated concentration) for 15 min at 37° C. Subsequently, 20 nM of 5′-end labeled substrate containing the abasic oligonucleotide (FIG. 6A) was added for 10 min at room temperature in reaction buffer described above for integrase catalytic assays. A freshly prepared solution of sodium borohydride (0.1 M final concentration) was added for 5 min. An equal volume (10 μl) of 2×SDS-polyacrylamide gel electrophoresis buffer (Invitrogen, Carlsbad, Calif.) was added in each reaction. Reaction products were heated at 95° C. for 1 min before analysis by electrophoresis in a 12-20% polyacrylamide gels (Invitrogen, Calif., USA). Gels were dried and reaction products were quantitated using the same method as described above.

Fluorimetric HIV-1 protease assay. The fluorescent HIV-1 protease substrate (RE-(EDANS)-SQNYPIVQK-(DABCYL)-R) was obtained from Molecular Probes, Inc. (Eugene, Oreg.). Substrate and buffer were pre-warmed at 37° C. for at least 20 min before use. The protease (25 nM final concentration) was incubated in the manufacturer's recommended assay buffer (100 mM Na acetate, 1M NaCl, 1 mM EDTA, 1 mM DTT, 10% DMSO and 1 mg ml⁻¹ BSA pH 4.7) at 37° C. in the presence of 1-25 μM NSC 18806 for 10 min and then added to the warmed substrate solution (40 μM) containing the different treatments to initiate the reaction. Acetyl pepstatin (Sigma, St. Louis, Mo.) at 20 nM was used a positive HIV-1 protease inhibitor control. The total assay volume was 100 □L. Fluorescence was monitored for 30 min in a fluorescence microplate reader (FMAX, Molecular Devices, Sunnyvale, Calif.) with 355 nm excitation and 460 nm emission filters and the rate of reactions compared for the different conditions.

Inhibition of HIV-induced cytopathic effect in cell culture. The MT-2 cells were grown in RPMI-1640 medium with GlutaMAX™, supplemented with 10% (v/v) heat-inactivated fetal bovine serum (both from Gibco, Invitrogen corporation, Carlsbad). The cells were maintained at 37° C. in a humidified atmosphere of 5% CO₂ in air. Every 4-5 days, cells were spun down and seeded at 2×10⁵ cells/ml in new cell culture flasks. HIV (HTLV-IBB isolate) was obtained from Advanced Biotechnology Incorporated (Columbia, Md.). The virus stock [3,2×10⁴ CCID₅₀ (50% cell culture infective dose) per ml as determined for MT-2 cells] was stored at −70° C. until used. Stock solutions of compounds were diluted using medium directly into 96-well assay plate (Costar, Corning inc, Corning, N.Y.).

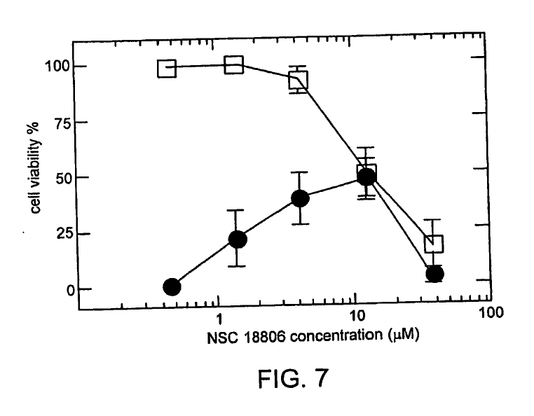

MT-2 cells (5×10⁵ cells/ml) were pretreated for 2 hours with test compounds at various concentrations as indicated in FIG. 7. Cells were then infected with 100 CCID₅₀ or mock-infected. The cell cultures were incubated at 37° C. in a humidified atmosphere of 5% CO₂ in air. Four days after infection the viability of mock- and HIV-infected cells was examined spectrophotometrically by the CellTiter 96 Non-Radioactive Cell proliferation assay (Promega, Madison, Wis.) and also confirmed microscopically in a hemacytometer by the trypan blue exclusion method. The percent cell viability in drug treated uninfected and infected cells was determined based on the viability of the uninfected control drug treated cells. The concentration of drug required to inhibit approximately 50% of the HIV-1 induced cytotoxicity was calculated from the plot of compound concentration verses the percent viable cells.

### Results

Inhibition of HIV-1 integrase by tropolone derivatives. The tropolone derivatives tested in this study are shown in Table 1. Compounds were first screened for inhibition of HIV-1 integrase in the presence of Mn²⁺ as a cofactor using DNA substrates mimicking the U5 LTR viral DNA end (FIG. 1A). The derivative containing only the minimal tropolone core structure (NSC 89303, Table 1) showed only marginal inhibition (IC₅₀>333 μM). Addition of an isopropyl group at positions 5 (NSC 43338), 4 (NSC 18804), 3 (NSC 18805) and of a 3-methyl-2-butenyl group at position 5 co-jointly with an isopropyl at the 4 position (NSC 43339) failed to increase potency. However, addition of an hydroxy group at position 7 (α-hydroxytropolone) resulted in inhibitory activity against REV-1 integrase (NSC 18806 and NSC 310618). NSC 18806 was the most inhibitory against integrase in strand transfer reaction (IC₅₀=4.8±2.5 μM) compared with NSC 310618 (IC₅₀=11.7±5.2 μM) (see Table 1).

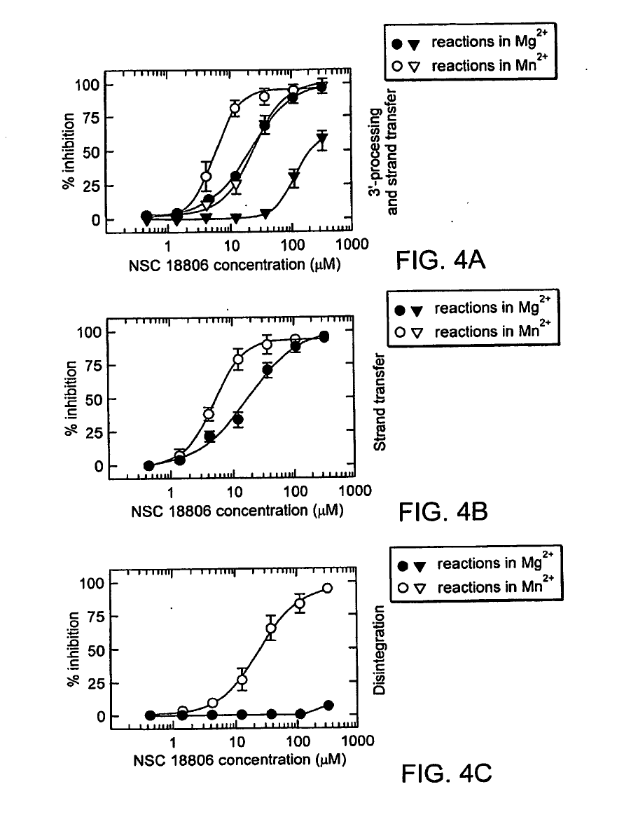

The tropolone NSC 18806 inhibits preferentially strand transfer in the presence of magnesium. For detailed characterization of NSC 18806, we compared its effect on the three reactions catalyzed by HIV integrase. 3′-processing, strand transfer and disintegration can be independently measured in biochemical assays using specific oligonucleotides (FIGS. 1A, 2A and 3A) (Marchand et al., 2001). A divalent cation, either Mg²⁺ or Mn²⁺, is required for integrase activity in vitro (Engelman and Craigie, 1995). Mg²⁺ is however the more likely cofactor in vivo. Because the integrase active site could be structurally different in the presence of Mg²⁺ or Mn²⁺ and inhibitors can act in different ways in Mg²⁺ or Mn²⁺ (Grobler et al., 2002; Marchand et al., 2003; Neamati et al., 2002), all assays were performed in the presence of either Mg²⁺ or Mn²⁺. FIGS. 1-3 show the results of representative experiments for the different assays and FIG. 4 and Table 1 summarize the results of these three assays.

NSC 18806 exhibited greater potency against 3′-P and ST using the standard 21 by oligonucleotide duplex in the presence of Mn²⁺ than in the presence Mg²⁺ (FIGS. 1B and 4, Table 1). The IC₅₀ values for 3′-P were approximately 5-fold higher than the IC₅₀ values for ST in the presence of either Mg²⁺ or Mn²⁺. Therefore NSC 18806 shows some selectivity for ST.

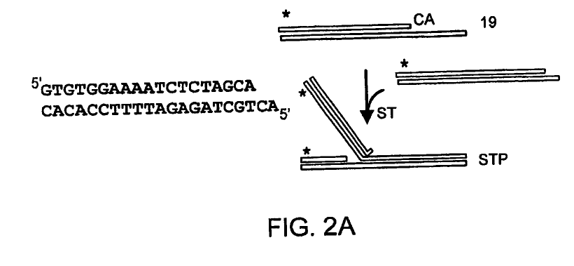

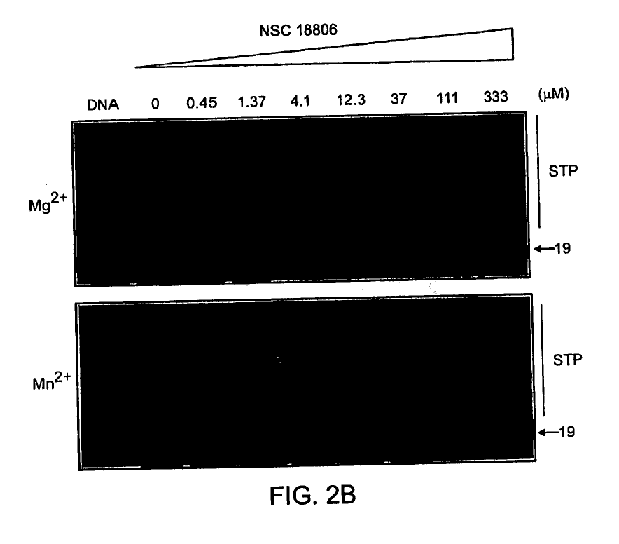

Because ST follows 3′-P in the reaction using the 21 by DNA substrate shown in FIG. 1, independent measurement of ST was performed with a preprocessed substrate (FIG. 2A). This assay segregates the action of a compound against ST from a decrease of the integrase activity related to the 3′-P inhibition in the overall integration. Results from this assay show similar ST inhibition and comparable IC₅₀ values for ST as were observed for overall integration (FIGS. 1B, 2B and 4, Table 1).

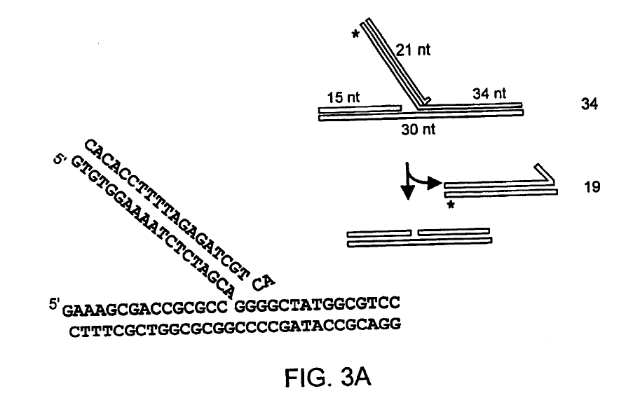

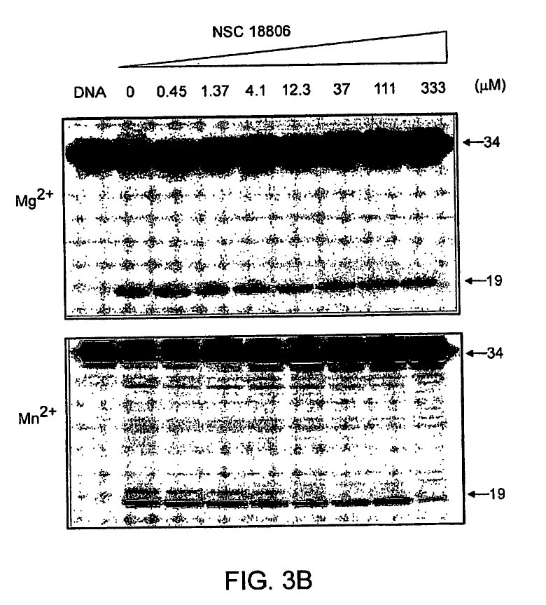

Disintegration was suggested as a reverse reaction of ST (Chow et al., 1992) (FIG. 3A). FIG. 3B shows the inability of NSC 18806 to inhibit disintegration in the presence of Mg²⁺. Disintegration was only inhibited in the presence of Mn²⁺ at high drug concentration. These results indicate that NSC 18806 is a more potent inhibitor of ST compared to disintegration (FIG. 4 and Table 1).

NSC 18806 affects the HIV-1 integrase catalytic site without inhibiting overall DNA binding. For determination of the possible binding site of NSC 18806 within the integrase catalytic site, we evaluated the ability of NSC 18806 to inhibit a crosslinking reaction between the cytosine in the 5′-AC overhang of the viral DNA and integrase glutamine 148 (FIG. 5A). A Q148C mutant form of HIV-1 integrase allows specific covalent interaction with a thiol-modified cytosine in the 5′-AC overhang without non-specific interference of other integrase cysteine residues (Johnson et al., 2005).

The results of this assay show metal-dependent inhibition of integrase-DNA disulfide-crosslinking by NSC 18806 (FIG. 5B). The importance of the α-hydroxy group in this inhibition is illustrated by the lack of inhibition observed for NSC 18804 (structural analog of NSC 18806 lacking the α-hydroxy group) (FIG. 5B). Although all reactions were performed with an equal concentration of enzyme, the origin of less integrase monomer in the gel after reaction with NSC 18806 was not determined. Concentration-dependent inhibition (FIG. 5C) suggests specific action of NSC 18806 against the crosslinking reaction. The IC₅₀ for crosslinking inhibition (32 μM) is comparable to the IC₅₀ for the ST inhibition in the presence of Mg²⁺ (21.6±3.40 μM, Table 1).

To determine whether crosslinking inhibition could be due to inhibition of overall binding of HIV-1 integrase to the viral DNA end, we investigated the ability of tropolone derivatives to inhibit crosslinking between integrase and a DNA substrate mimicking viral U5 LTR end and containing an abasic site corresponding to the adenine in the conserved CA-dinucleotide (FIG. 6A). NSC 18806 did not block the Schiff base IN-DNA interaction (FIG. 6B) indicating specific inhibition of disulfide crosslinking.

NSC 18806 exhibits moderate cytoprotective activity against HIV-1 in cell-based assay. The tropolone compounds shown in Table 1 were tested in an HIV infectivity assay (Pauwels et al., 1988). All compounds demonstrated at least weak activity in this assay. NSC 18806 showed protection of infected cells from HIV-induced cytopathic effect with an estimated IC₅₀ of approximately 12 μM (FIG. 7). The IC₅₀ could only be estimated due to the presence of toxicity at concentrations at and above 12 μM. Note, that the cytoprotective concentration for this compound is comparable to the IC₅₀ for integrase inhibition in vitro in the presence of Mg²⁺.

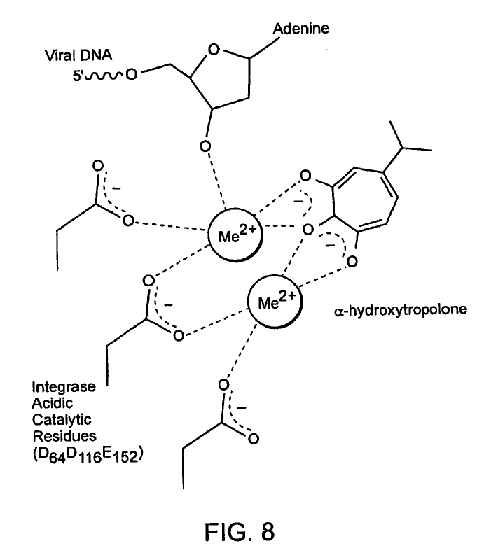

Tropolone derivatives have a range of antimicrobial activities. They are antifungal (Baya et al., 2001), antibacterial (Morita et al., 2004) and insecticidal (Morita et al., 2003). They also exhibit antioxidant properties (Doulias et al., 2005). Recently, specific inhibition of the RNaseH domain of HIV-1 reverse transcriptase by 7-hydroxytropolone derivatives (α-hydroxytropolones) was reported (Budihas et al., 2005). These various biological activities of tropolone derivatives have been linked with their ability to chelate metals (Budihas et al., 2005; Doulias et al., 2005; Matsumura et al., 2001). The current study suggests that α-hydroxytropolones may also inhibit HIV-1 integrase by chelation of the Mg²⁺ or Mn²⁺ in the enzyme active site (FIG. 8).

According to the two crosslinking assays, α-hydroxytropolones interfere with the protruding 5′-end of the LTR and the integrase amino group glutamine 148 in the integrase flexible loop (inhibition of disulfide crosslinking) without affecting the overall binding of integrase to the viral DNA end (no inhibition of Schiff base crosslinking). It has been suggested that the interaction between the cytosine in the 5′ overhang of the viral DNA and the Q148 occurs after a conformational change of the integrase-viral (donor) DNA complex, which is necessary for triggering ST (Johnson et al., 2005). Thus, the crosslinking results coupled with the ability of α-hydroxytropolones to preferentially inhibit ST (full-length or preprocessed substrate) compared to 3′-P in the presence of Mg²⁺ suggest preferential binding of the α-hydroxytropolones to the integrase-DNA complex following 3′-P.

The lack of inhibition of the disintegration reaction by α-hydroxytropolones in the presence of Mg²⁺ compared with effective inhibition of the ST reaction is noticeable because disintegration corresponds to the reverse reaction of strand transfer (Chow et al., 1992). The same selectivity for strand transfer vs. disintegration was shown for the diketo acid derivative L-731,988, and led to the interpretation that diketo acids bind to the target DNA site (Espeseth et al., 2000). Competition with target (acceptor) DNA could explain why NSC 18806 has a lower affinity for binding to the integrase catalytic active site if this site is already occupied by the donor and acceptor DNA, which would be the case for the disintegration substrate. Hence α-hydroxytropolones might act as interfacial inhibitors (Pommier and Cherfils, 2005; Pommier and Marchand, 2005) for HIV-1 integrase-divalent metal-DNA complexes and block the binding of the acceptor (genomic) DNA.

α-hydroxytropolones were more potent but less selective for ST in the presence of Mn²⁺ than in the presence of Mg²⁺. Such metal-dependent inhibition might be due to a different folding of the integrase active site in the presence of Mg²⁺ or Mn²⁺. Mn²⁺ is geometrically wider than Mg²⁺ (Bock et al., 1999; Huang et al., 1997) and therefore the catalytic site of integrase could be more “open” in the presence of Mn²⁺, which might allow the α-hydroxytropolones to enter various conformations of this site.

NSC 18806 shows cytoprotective activity on HIV-infected cells, which appears limited by the cytotoxicity of the drug. The cytoprotection against virus could be due to drug action against other steps beside HIV replication. However, we can exclude HIV protease as NSC 18806 failed to inhibit HIV-1 protease at concentration up to 25 μM in a standard fluorescent-based HIV-1 protease assay (data not shown). Recently, it was reported that the α-hydroxytropolones inhibit the RNaseH domain of HIV-1 reverse transcriptase (Budihas et al., 2005). The topological similarity between the catalytic domain of HIV integrase and the HIV reverse transcriptase RNase H domain could indicate a common mechanism of metal chelation in the enzyme catalytic site(s) at work (Dyda et al., 1994; Yang and Steitz, 1995).

All documents (including patents, patent applications and literature references) mentioned herein are incorporated herein by reference.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

### REFERENCES

Asante-Appiah E and Skalka A M (1997) Molecular mechanisms in retrovirus DNA integration. Antiviral Res 36(3):139-156.
Barbaro G, Scozzafava A, Mastrolorenzo A and Supuran C T (2005) Highly active antiretroviral therapy: current state of the art, new agents and their pharmacological interactions useful for improving therapeutic outcome. Curr Pharm Des 11(14):1805-1843.
Baya M, Soulounganga P, Gelhaye E and Gerardin P (2001) Fungicidal activity of beta-thujaplicin analogues. Pest Manag Sci 57(9):833-838.
Bock C W, Katz A K, Markham G D and Glusker J P (1999) Manganese as a replacement for magnesium and zinc: functional comparison of the divalent ions. J Am Chem Soc 121:7360-7372.
Brown P O (1990) Integration of retroviral DNA. Curr Top Microbiol Immunol 157:19-48.
Budihas S R, Gorshkova I, Gaidamakov S, Wamiru A, Bona M K, Parniak M A, Crouch R J, McMahon J B, Beutler J A and Le Grice S F (2005) Selective inhibition of HIV-1 reverse transcriptase-associated ribonuclease H activity by hydroxylated tropolones. Nucleic Acids Res 33(4):1249-1256.
Chiu T K and Davies D R (2004) Structure and function of HIV-1 integrase. Curr Top Med Chem 4(9):965-977.
Chow S A, Vincent K A, Ellison V and Brown P O (1992) Reversal of integration and DNA splicing mediated by integrase of human immunodeficiency virus. Science. 255(5045):723-726.
De Clercq E (2005) New approaches toward anti-HIV chemotherapy. J Med Chem 48(5):1297-1313.
Debyser Z, Cherepanov P, Van Maele B, De Clercq E and Witvrouw M (2002) In search of authentic inhibitors of HIV-1 integration. Antivir Chem Chemother 13(1):1-15.
Deprez E, Barbe S, Kolaski M, Leh H, Zouhiri F, Auclair C, Brochon J C, Le Bret M and Mouscadet J F (2004) Mecbanism of HIV-1 integrase inhibition by styrylquinoline derivatives in vitro. Mol Pharmacol 65(1):85-98.
Diouf P N, Delbarre N, Perrin D, Gerardin P, Rapin C, Jacquot J P and Gelhaye E (2002) Influence of tropolone on Poria placenta wood degradation. Appl Environ Microbiol 68(9):4377-4382.
Doulias P T, Nousis L, Zhu B Z, Frei B and Galaris D (2005) Protection by tropolones against H2O2-induced DNA damage and apoptosis in cultured Jurkat cells. Free Radic Res 39(2):125-135.
Dyda F, Hickman A B, Jenkins T M, Engelman A, Craigie R and Davies D R (1994) Crystal structure of the catalytic domain of HIV-1 integrase: similarity to other polynucleotidyl transferases. Science 266(5193):1981-1986.
Engelman A and Craigie R (1992) Identification of conserved amino acid residues critical for human immunodeficiency virus type 1 integrase function in vitro. J Virol 66(11):6361-6369.
Engelman A and Craigie R (1995) Efficient magnesium-dependent human immunodeficiency virus type 1 integrase activity. J Virol 69(9):5908-5911.
Espeseth A S, Felock P, Wolfe A, Witmer M, Grobler J, Anthony N, Egbertson M, Melamed J Y, Young S, Hamill T, Cole J L and Hazuda D J (2000) HIV-1 integrase inhibitors that compete with the target DNA substrate define a unique strand transfer conformation for integrase. Proc Natl Acad Sci USA 97(21):11244-11249.
Fesen M R, Kohn K W, Leteurtre F and Pommier Y (1993) Inhibitors of human immunodeficiency virus integrase. Proc Natl Acad Sci USA 90(6):2399-2403.
Goldgur Y, Craigie R, Cohen G H, Fujiwara T, Yoshinaga T, Fujishita T, Sugimoto H, Endo T, Murai H and Davies D R (1999) Structure of the HIV-1 integrase catalytic domain complexed with an inhibitor: a platform for antiviral drug design. Proc Natl Acad Sci USA 96(23):13040-13043.
Grobler J A, Stillmock K, Hu B, Witmer M, Felock P, Espeseth A S, Wolfe A, Egbertson M, Bourgeois M, Melamed J, Wai J S, Young S, Vacca 7 and Hazuda D J (2002) Diketo acid inhibitor mechanism and HIV-1 integrase: implications for metal binding in the active site of phosphotransferase enzymes. Proc Natl Acad Sci U S A 99(10):6661-6666.
Hazuda D J, Felock P, Witmer M, Wolfe A, Stilimock K., Grobler J A, Espeseth A, Gabryelski L, Schleif W, Blau C and Miller M D (2000) Inhibitors of strand transfer that prevent integration and inhibit HIV-1 replication in cells. Science 287(5453):646-650.
He C and Verdine G L (2002) Trapping distinct structural states of a protein/DNA interaction through disulfide crosslinking. Chem Biol 9(12):1297-1303.
Huang Y, Beaudry A, McSwiggen J and Sousa R (1997) Determinants of ribose specificity in RNA polymerization: effects of Mn2+ and deoxynucleoside monophosphate incorporation into transcripts. Biochemistry 36(44):13718-13728.
Johnson A, Santos W, Marchand C, Amin R, Verdine G and Pommier Y (2005) Retroviral integration requires the viral DNA end to interact with the integrase flexible loop. J. Biol. Chem. 10.1074/jbc.M511348200.
Johnson A A, Marchand C and Pommier Y (2004) HIV-1 integrase inhibitors: a decade of research and two drugs in clinical trial. Curr Top Med Chem 4(10):1059-1077.
Kellerman S, Begley E, Boyett B, Clark H and Schulden J (2005) Changes in HIV and AIDS in the United States: Entering the Third Decade. Curr Infect Dis Rep 7(2):138-143.
Leh H, Brodin P, Bischerour J, Deprez E, Tauc P, Brochon J C, LeCam E, Coulaud D, Auclair C and Mouscadet J F (2000) Determinants of Mg2+-dependent activities of recombinant human immunodeficiency virus type 1 integrase. Biochemistry 39(31):9285-9294.
Lim Y W, Kim J J, Chedgy R, Morris P I and Breuil C (2005) Fungal diversity from western redcedar fences and their resistance to beta-thujaplicin. Antonie Van Leeuwenhoek 87(2):109-117.
Marchand C, Johnson A A, Karki R G, Pais G C, Zhang X, Cowansage K, Patel T A, Nicklaus M C, Burke T R, Jr. and Pommier Y (2003) Metal-dependent inhibition of HIV-1 integrase by beta-diketo acids and resistance of the soluble double-mutant (F185K/C280S). Mol Pharmacol 64(3):600-609.
Marchand C, Neamati N and Pommier Y (2001) In vitro human immunodeficiency virus type 1 integrase assays. Methods Enzymol 340:624-633.
Matsumura E, Morita Y, Date T, Tsujibo H, Yasuda M, Okabe T, Ishida N and Inamori Y (2001) Cytotoxicity of the hinokitiol-related compounds, gamma-thujaplicin and beta-dolabrin. Biol Pharm Bull 24(3):299-302.
Mazumder A and Pommier Y (1995) Processing of deoxyuridine mismatches and abasic sites by human immunodeficiency virus type-1 integrase. Nucleic Acids Res 23(15):2865-2871.
Morita Y, Matsumura E, Okabe T, Fukui T, Ohe T, Ishida N and Inamori Y (2004) Biological activity of beta-dolabrin, gamma-thujaplicin, and 4-acetyltropolone, hinokitiol-related compounds. Biol Pharm Bull 27(10):1666-1669.
Morita Y, Matsumura E, Okabe T, Shibata M, Sugiura M, Ohe T, Tsujibo H, Ishida N and Inamori Y (2003) Biological activity of tropolone. Biol Pharm Bull 26(10):1487-1490.
Neamati N, Hong H, Sunder S, Milne G W and Pommier Y (1997) Potent inhibitors of human immunodeficiency virus type 1 integrase: identification of a novel four-point pharmacophore and tetracyclines as novel inhibitors. Mol Pharmacol 52(6):1041-1055.
Neamati N, Lin Z, Karki R G, Orr A, Cowansage K, Strumberg D, Pais G C, Voigt J H, Nicklaus M C, Winslow H E, Zhao H, Turpin J A, Yi J, Skalka A M, Burke T R, Jr. and Pommier Y (2002) Metal-dependent inhibition of HIV-1 integrase. J Med Chem 45(26):5661-5670.
Pauwels R, Balzarini J, Baba M, Snoeck R, Schols D, Herdewijn P, Desmyter J and De Clercq E (1988) Rapid and automated tetrazolium-based colorimetric assay for the detection of anti-HIV compounds. J Virol Methods 20(4):309-321.
Pommier Y and Cherfils J (2005) Interfacial inhibition of macromolecular interactions: nature's paradigm for drug discovery. Trends Pharmacol Sci 26(3):138-145.
Pommier Y, Johnson A A and Marchand C (2005) Integrase inhibitors to treat HIV/AIDS. Nat Rev Drug Discov 4(3):236-248.
Pommier Y and Marchand C (2005) Interfacial inhibitors of protein-nucleic acid interactions. Curr Med Chem Anti-Canc Agents 5(4):421-429.
Turner B G and Summers M F (1999) Structural biology of HIV. J Mol Biol 285(1):1-32.
Van Maele B and Debyser Z (2005) HIV-1 integration: an interplay between HIV-1 integrase, cellular and viral proteins. AIDS Rev 7(1):26-43.
Yang W and Steitz T A (1995) Recombining the structures of HIV integrase, RuvC and RNase H. Structure 3(2):131-134.

From the above description, one skilled in the art can easily ascertain the essential characteristics of the present invention, and without departing from the spirit and scope thereof, can make various changes and modifications of the invention to adapt it to various usages and conditions. Thus, other embodiments are within the appended claims.

1. A method of treating HIV infection in a subject comprising identifying a subject as in need of inhibition of HIV integrase; and administering an effective amount of one or more tropolone compounds, or salt, solvate or hydrate thereof.

2. The method of claim 1 wherein one or more α-hydroxytropolone compounds are administered.

3. The method of claim 1 wherein one or more mono-hydroxytropolone compounds are administered.

4. The method of claim 1 wherein one or more bis-hydroxy tropolone compounds are administered.

5. The method of claim 1 wherein one or more compounds of the following Formula (I) are administered 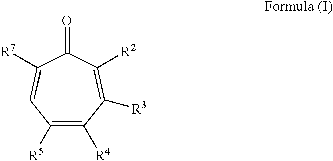
wherein,
each R¹ is independently F, Cl, Br, CF₃, NH₂, N(C₁-C₆ alkyl)₂, NO₂, CN, (C₁-C₆ alkyl)O—, —OH, (C₁-C₆ alkyl)S(O)ₘ—, (C₁-C₆ alkyl)C(O)NH—, H₂N—C(NH)—, (C₁-C₆ alkyl)C(O)—, (C₁-C₆ alkyl)OC(O)—, N₃, (C₁-C₆ alkyl)OC(O)NR— and C₁-C₂₀ alkyl;
each R² is independently OH;
each R³ is independently H, alkyl, or R³ taken together with R⁴ and the carbon atoms to which they are each attached, respectively, form a cycloalkyl which may be optionally substituted with 1-4 R¹;
each R⁴ is independently H, alkyl, or R⁴ taken together with R³ and the carbon atoms to which they are each attached, respectively, form a cycloalkyl which may be optionally substituted with 1-4 R¹;
each R⁵ is independently H, alkyl or alkenyl;
each R⁷ is independently H or OH; and
each m is independently 0, 1 or 2.

6. The method of claim 5 wherein the integrase inhibitor is one or more of the compounds of Formula (I) or salt, solvate or hydrate thereof, wherein R⁷ is OH.

7. The method of claim 1 wherein the one or more tropolone compounds comprise one or more of the compounds of Table 1.

8-9. (canceled)

10. A method of inhibiting HIV replication in a cell or a subject comprising identifying a subject as in need of HIV-1 integrase inhibition in HIV-infected cells; and administering an effective amount of one or more tropolone compounds, or salt, solvate or hydrate thereof.

11. A method of treating HIV-infected cells in a subject comprising identifying a subject as in need of inhibition of HIV integrase; and administering an effective amount of one or more tropolone compounds, or salt, solvate or hydrate thereof.

12. A method of modulating strand transfer (ST) in an HIV-infected cell in a subject identified as in need of such treatment comprising administering to the subject of an effective amount of one or more tropolone compounds, or salt, solvate or hydrate thereof.

13. The method of claim 10 wherein the cell is a lymphocytic cell.

14. The method of claim 10 wherein the cell is a monocytic cell.

15. The method of claim 10 wherein the cells are human cells.

16. The method of claim 10 wherein one or more α-hydroxytropolone compounds or one or more bis-hydroxy tropolone compounds are administered.

17. The method of claim 10 wherein one or more mono-hydroxytropolone compounds are administered.

18. (canceled)

19. The method of claim 10 wherein one or more compounds of the following Formula (I) are administered 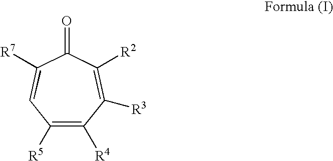
wherein,
each R¹ is independently F, Cl, Br, CF₃, NH₂, N(C₁-C₆ alkyl)₂, NO₂, CN, (C₁-C₆ alkyl)O—, —OH, (C₁-C₆ alkyl)S(O)ₘ—, (C₁-C₆ alkyl)C(O)NH—, H₂N—C(NH)—, (C₁-C₆ alkyl)C(O)—, (C₁-C₆ alkyl)OC(O)—, N₃, (C₁-C₆ alkyl)OC(O)NR— and C₁-C₂₀ alkyl;
each R² is independently OH;
each R³ is independently H, alkyl, or R³ taken together with R⁴ and the carbon atoms to which they are each attached, respectively, form a cycloalkyl which may be optionally substituted with 1-4 R′;
each R⁴ is independently H, alkyl, or R⁴ taken together with R³ and the carbon atoms to which they are each attached, respectively, form a cycloalkyl which may be optionally substituted with 1-4 R¹;
each R⁵ is independently H, alkyl or alkenyl;
each R⁷ is independently H or OH; and
each m is independently 0, 1 or 2.

20. (canceled)

21. A method of inhibiting proviral DNA insertion into a host chromosome in an HIV-infected subject identified as in need of such treatment comprising administering to the subject of an effective amount of one or more of one or more tropolone compounds, or salt, solvate or hydrate thereof.

22. (canceled)

23. The method of claim 1 further comprising administration of one or more additional anti-HIV therapeutic agents.

24. The method of claim 23 wherein the additional agent(s) are a reverse transcriptase inhibitor, a protease inhibitor, an integrase inhibitor, or combination thereof.

25. A method of inhibiting HIV-1 integrase in a HIV-infected cell or subject comprising administration to the cell or subject of an effective amount of one or more of one or more tropolone compounds, or salt, solvate or hydrate thereof such that the inhibition is mediated by chelation of Mg²⁺ or Mn²⁺ in the enzyme active site.

26-29. (canceled)


SEQUENCE LISTING (7 sequences)

SEQ ID NO: 1 - DNA, 21 bp, Artificial Sequence
  Oligonucleotide primer
gtgtggaaaatctctagcagt

SEQ ID NO: 2 - DNA, 21 bp, Artificial Sequence
  Oligonucleotide primer
actgctagagattttccacac

SEQ ID NO: 3 - DNA, 19 bp, Artificial Sequence
  Oligonucleotide primer
gtgtggaaaatctctagca

SEQ ID NO: 4 - DNA, 30 bp, Artificial Sequence
  Oligonucleotide primer
gaaagcgaccgcgccggggctatggcgtcc

SEQ ID NO: 5 - DNA, 30 bp, Artificial Sequence
  Oligonucleotide primer
ctttcgctggcgcggccccgataccgcagg

SEQ ID NO: 6 - DNA, 20 bp, Artificial Sequence
  Oligonucleotide primer
gtgtggaaaatctctagcgt

SEQ ID NO: 7 - protein, 9 aa, Artificial Sequence
  Synthetic construct
SQNYPIVQK